(19) United States
(12) Patent Application Publication
(10) Pub. No.: US 2024/0100164 A1
(43) Pub. Date: Mar. 28, 2024

(54) REPEATED PHOTODYNAMIC THERAPY USING PHOTOSENSITIZING AGENT

(71) Applicant: Incando Therapeutics Pte. Ltd., Singapore (SG)

(72) Inventor: Percy Luu, Singapore (SG)

(21) Appl. No.: 18/372,423

(22) Filed: Sep. 25, 2023

Related U.S. Application Data
(60) Provisional application No. 63/410,683, filed on Sep. 28, 2022.

(51) Int. Cl. A61K 41/00; A61N 5/06; A61P 35/00

(52) U.S. Cl. CPC A61K 41/0061; A61N 5/0601; A61N 5/062; A61P 35/00; A61N 2005/0626; A61N 2005/0651; A61N 2005/0659

(57) ABSTRACT

A phototherapy method includes delivering a photosensitizer to a patient to accumulate in target treatment tissue. The target treatment tissue is illuminated with light from an illumination source in a wirelessly powered illumination device thereby photoactivating the photosensitizing agent. The combination of oxygen in cells of the target treatment tissue and the photoactivated photosensitizer generates cytotoxic reactive oxygen species which destroys cells in the target treatment tissue.

### CLAIM OF PRIORITY

The present patent application claims the benefit of priority of U.S. Provisional Patent Application No. 63/410,683 (Attorney Docket No. 5405.003PRV), filed on Sep. 28, 2022; the entire contents of which are incorporated herein by reference.

### BACKGROUND

Light delivery as a therapeutic is an integral part of human existence. Light from the sun helps regulate our circadian rhythm and produce crucial Vitamin D in our skin throughout the day. Light is used in the form of therapy to treat conditions of the eyes and skin, or to reduce bilirubin levels to treat newborn jaundice. Light delivery may be used to treat a number of other conditions.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an example of an illumination device coupled to a patient's head.

FIG. 2 shows an example of an illumination device implanted in the brain.

FIG. 3 illustrates an example of an illumination device.

FIG. 4 shows an example of a power delivery device.

FIG. 5 shows another example of a power delivery device.

FIG. 6 shows an example of a head strap that may be used to hold an illumination device and/or power delivery device against a patient's head.

FIG. 7 shows the chemical structure of 5-amino-4-oxo-pentanoic acid hydrochloride.

FIG. 8 shows the chemical structure of Protoporphyrin IX (PPIX).

FIGS. 9A-9B show in vitro data related to PDT induced cell death of glioma cells using a light source.

FIG. 10 shows rat C6 glioma cells implanted into striatum of rats.

FIGS. 11A-11E show an implantable illumination device used to treat GBM in rodents.

FIGS. 12A-12C show an illumination treatment schema and survival in an orthotopic GGM model.

FIG. 13 shows survival curves for single vs repeat PDT treated animals.

FIG. 14 shows minimal effect produced by an implanted illumination device on an MRI image.

### DETAILED DESCRIPTION

The main challenge of using light to treat internal diseases is that light does not travel very far into the body. Light is absorbed by the skin and tissue, which limits the penetration depth of visible and near-infrared. (NIR) wavelengths to 3 to 5 millimeters. Applying phototherapy to tumors or stimulating neurons to treat movement disorders or other neurodegenerative diseases may require light at 10-25 centimeter depths, less depth, or even greater depths, and an implantable light source adjacent the target treatment area is the only practical way for light to reach such depths in the human body.

This disclosure describes powering, light delivery, and integration aspects of an implantable phototherapy device, system and methods of use. The implant can also be designed to deliver a single treatment and/or integrated with other forms of stimulation andlor therapeutic agents other than light, and in doing so deliver innovative combination therapies. The implant can be used to photoactivate one or more photosensitizing agents.

### Example Device for Photodynamic Therapy (PDT)

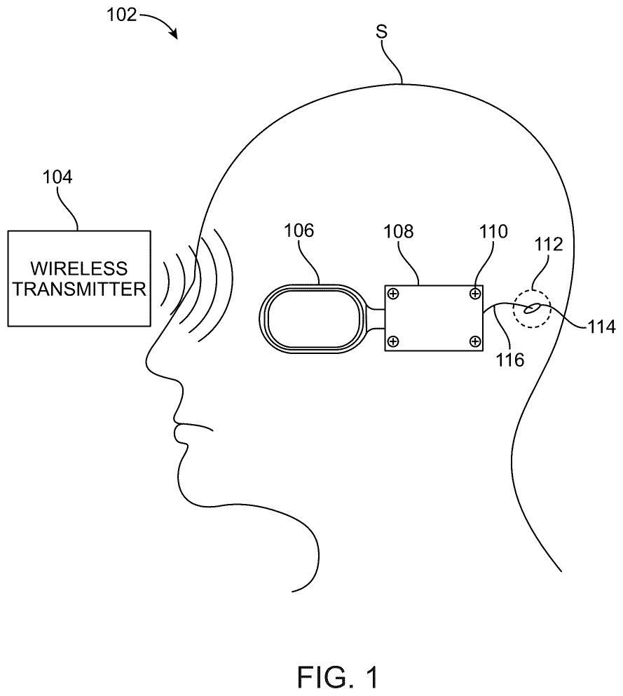

FIG. 1 shows shows an example of a phototherapy system 102 with an external power source 104 which provides radiofrequency power wirelessly to the phototherapy system. The device may be used to provide single or repeated photodynamic therapy. The phototherapy device includes a power receiver element that includes a coil 106 for wirelessly receiving radiofrequency energy (RF) energy from the power source 104, here a RF wireless transmitter. The power receiver element also includes a housing 108 that contains the electronic components for controlling the phototherapy system. Fasteners 110 such as screws maybe be used to secure the housing to the skull S under the scalp. A tether 116 electrically and mechanically couples the housing and the electronic components in the housing with the illumination element (not seen) that is disposed in a cavity in the brain tissue formed after the tumor has been resected. The bone plate 112 may be repositioned in the burr hole formed in the skull S to help close the skull and a grommet 114 or clip may be used to help secure the tether to the skull and prevent damage to the tether. The tether may be coiled or uncoiled. Here the power receiver element may be positioned on a side of the patient's head, about eye level. The housing may be formed from any biocompatible material such as titanium and provides a hermetic seal for the electronic components. The housing may serve as a heat dissipation element, or a separate heat dissipation element (not shown) may also be included in the housing. Electrical leads exiting the housing form the tether 116 which is coupled to the illumination source to deliver power to the illumination source and optionally transfer data to and from the illumination source or otherwise control the illumination source. The tether may also serve as a mechical connector between the illumination source and the housing to prevent separation and migration of the light source away from the rest of the device. While this device is described primarily for the use of treating gliobastoma multiforme (GBM), this is not intended to be limiting and the device may be used to provide illumination in other diseases or conditions requiring light, such as neurodegenerative diseases.

Neurodegenerative diseases are a group of conditions that cause the progressive loss of nerve cells in the brain and/or spinal cord. This damage can lead to a variety of symptoms, including memory loss, difficulty thinking or reduction of cognitive function, movement problems, and behavioral changes. There are over 50 known neurodegenerative diseases, some of the most common include Alzheimer's disease, Parkinson's disease, Huntington's disease, multiple sclerosis, Amyotrophic lateral sclerosis (ALS), Creutzfeldt-Jakob disease (CJD), Friedreich's ataxia, and Multiple system atrophy (MSA), dyskinesia.

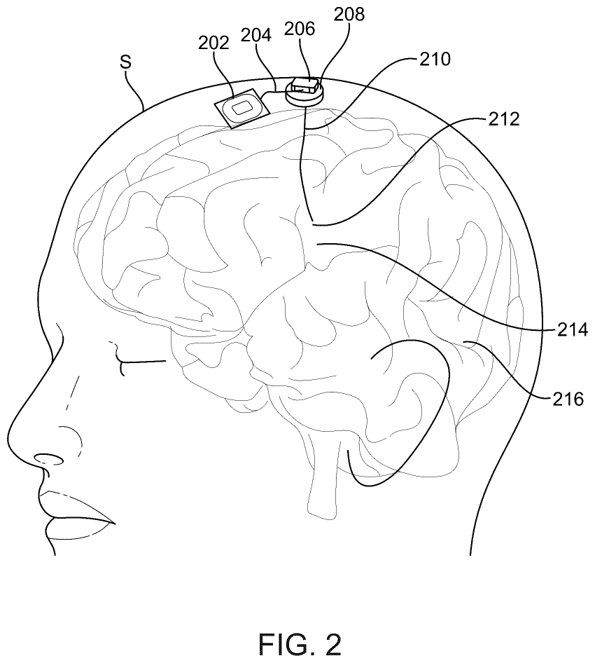

FIG. 2 more clearly illustrates implantation of the illumination device such as the device of FIG. 1 in the target treatment tissue, here the brain 216. The device that is implanted delivers light energy and is wirelessly powered. It may be surgically implanted within the resection cavity created during or after a debulking surgery for a brain tumor such as a glioblastoma multiforme (GBM) or any other tumor in the brain or tumors elsewhere in the body, or even any diseased or damaged tissue in the body. It may also be surgically implanted without the requirement of any debulking surgery in target treatment tissue at or near the treatment site. The implant consists of several parts including a light source 212 such as an LED light capable of illuminating the resection cavity or nearby tissue with a desired wavelength of light, such as near infrared light, e.g. 635 nm, a tether wire 210 that leads from the LED light source to a separate electronics enclosure 206 that may contain power regulating circuitry and a power receiving coil 202, implanted under the skin and atop the skull S. The tether wire may pass through a burr hole in the skull S that may be sealed with a burr hole cap 208 and an optional grommet. The LED emits light 214 to target treatment tissue. A second wire 204 electrically couples the power receiving coil with the electronics enclosure and the second wire may be integral with the tether 201 or may be a separate wire.

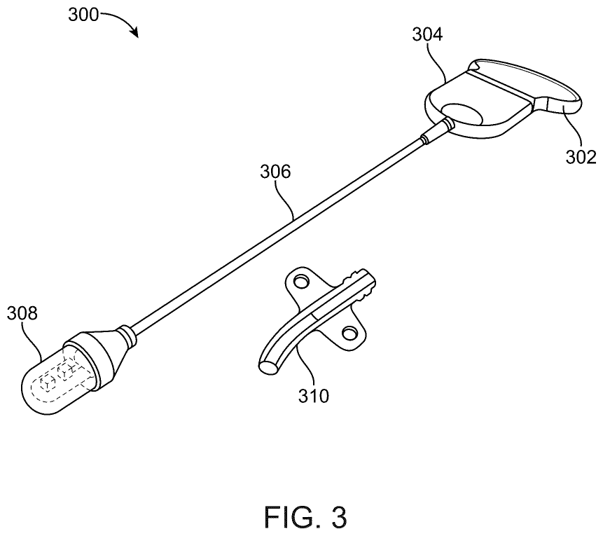

FIG. 3 illustrates an example of an implantable light delivery device 300 that may be used to treat GBM, any other tumors, or other conditions such as neurodegenerative diseases. The device 300 may include a receiver coil 302, a sealed housing 304 with electronic components, a tether 306, the light source 308 and an optional suture wing 310 which may be coupled anywhere along the tether. Bone screws and a burr cap and grommet may also be supplied with the device in kit format to allow the device to be secured to a patient's skull. The tether may be an elongate wire or filament such that the length of the tether is significantly longer than the length of other components to allow the light source to reach deep into the brain if needed.

As previously mentioned, although a resection surgery is not required for implantation, the device may be implanted as part of a standard tumor debulking surgery for GBM or other tumors. The light source emits light which either has a therapeutic effect on the target tissue or the light may be used to activate a drug which has a therapeutic effect on the target tissue. Examples of wavelengths of emitted light and light sources are disclosed throughout this specification and may be used in this or any example of the device. The tether allows power to be transferred from the receiver coil to the housing with electronics and then to the light source. The housing with electronics contains electronic components which control operation of the device. In some examples, the tether may also allow data to be transferred to/from the light source and the housing with electronics. The tether may also provide a physical connection between the light and the housing with electronics so that the light is held and does not migrate once implanted, thus the light remains implanted at the site of the resection cavity after removal of the GBM tumor. The housing with the electronics and the receiver coil may be implanted under the skin and attached to the skull.

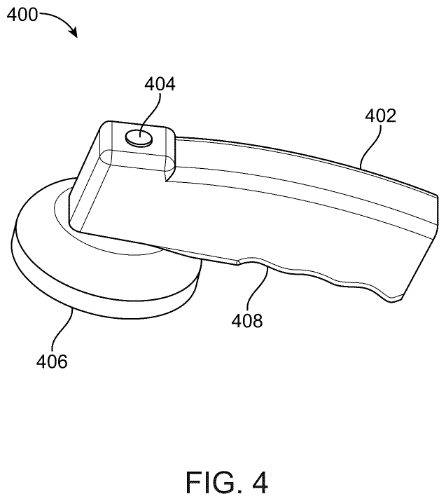

FIG. 4 illustrates an example of a surgical power delivery device 400 that may be used with any of the illumination devices disclosed herein, such as in FIG. 3 above. The surgical power delivery device may be used intraoperatively in the operating theater during surgery, immediately after surgery, or post-operatively in the clinic or at home. The power delivery device may be used to deliver power externally from the patient through the skin to the power receiver coil on the illumination device which then can power the light source and associated electronics while the power delivery system delivers energy, or the energy may be stored for later use by batteries in the illumination device.

The device 400 may include a handle 402, a power on/off button 404 with optional indicator light, and a transmitter coil 406. Batteries may be disposed in the handle (not illustrated) to provide power, or the handle may be plugged into an outlet or house mains to provide the power. The handle is ergonomically shaped so that the handheld device is comfortably grasped and manipulated by an operator and may include optional fmger recesses 408 in the handle to provide a comfortable hand grip. The transmitter coil may also be ergonomically shaped to conform comfortably to the patient's head and also with the receiver coil on the device. In use, the operator may hold the power delivery device up against the patient's head and adjacent to the power receiver coil in the illumination device to provide it with power. If used intraoperatively, a sterile plastic bag or other cover may be disposed over the power delivery device. If used in the clinic, physician's office or at home, the operator may simply grasp the device in their hand and bring it into apposition with the patient's head.

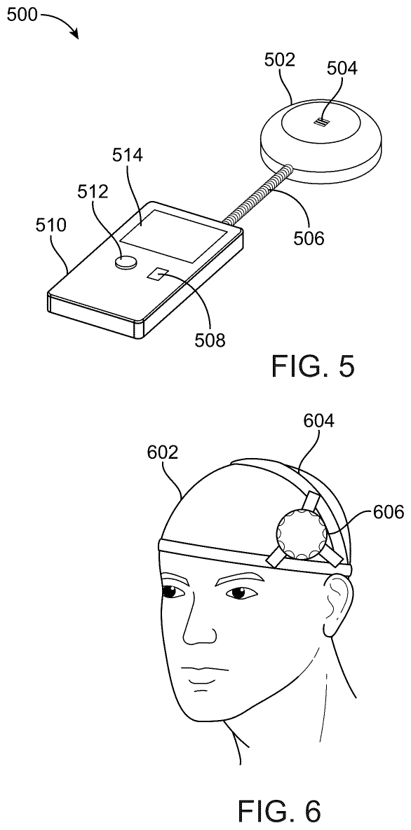

FIG. 5 shows another example of a power delivery device 500 that may be used to deliver power to the illumination device. The power delivery device may be used intraoperatively in the operating theater or post-operatively in the clinic or at home. The device may have a housing 510 with an on/off switch (not illustrated), a run/pause button 512, a display 514, and a battery level indicator 508. A flexible cable 506 electrically couples the housing 510 with a puck 502 or power delivery housing that has a delivery coil (not shown) and optionally a proximity indicator such as light bar 504. The proximity indicator helps the operator align the power delivery device with the receiver coil disposed under the patient's scalp. In some examples, the device may be powered from a wall socket or house mains instead of a battery, or to recharge the batteries, or a battery may be used. The batteries may be a backup power supply and may be disposable or rechargeable.

Actuating the on/off switch (not shown) turns the device on or off. Actuation of the run/pause button 512 allows power to be delivered to the illumination device or paused. The battery level indicator shows how much energy is left in the batteries (not shown) in the housing 510. A display 514 can show various parameters such as the amount of time that energy is delivered to the illumination device, system status, or any other desired information. The cable 506 allows power from the housing 510 to be delivered to the delivery coil (not shown) in the puck 502. Alignment of the delivery coil with the receiver coil in the illumination device may be monitored using the proximity light bar 504. When the coils are properly aligned, the proximity light bar will indicate a good electromagnetic connection between the two components, and when they are not properly aligned, the light bar will indicate that further adjustment is needed. The puck may be advanced into apposition or near apposition with the patient's skull so that power can then be transferred from the power delivery device to the illumination device to allow the light to illuminate the target treatment tissue with the desired wavelength of light for a desired time period, or to charge a battery in the illumination device for later therapy.

The power delivery device may optionally have other components as desired, such as a speaker to provide audible feedback to either the patient or operator. Barrel connectors may be used to allow one component to be mechanically and electrically coupled with another component.

FIG. 6 shows an optional headband 604 that may be used to hold the power delivery coil 606 from a power delivery device against the patient's head 602 and adjacent the power receiver coil (disposed under the scalp, not seen in this view) so the operator can use their hands for other purposes during the treatment. Power is then wirelessly transferred from the power delivery device to the illumination device.

### Light Therapy

A wirelessly powered implantable light illumination system such as any of those described herein may be used to photoactivate a photosensitizing agent. Photosensitizing agents include, but are not limited to certain porphyrins and related tetrapyrrole compounds. Most photosensitizing agents are based on a porphyrin structure, such as benzoporphyrins, purpurins, texaphyrins, phthalocyanines, naphthalocyanines, and protoporphyrin IX (PPIX). PPIX is a precursor of heme and is involved in the metabolism of heme through the combination of mitochondrial transport proteins. Another commonly used photosensitizer is 5-aminolevulinic acid (5-ALA, or sometimes also referred to herein as ALA), the biological precursor of PPIX. Other photosensitizers, like mono-aspartyl chlorin e6 (NPe6), and hexylpyropheophorbide (HPPH), are based on the chlorin structure. Other second-generation photosensitizers were designed to meet specific demands, such as the mitochondria-targeting photosensitizers, DLC (delocalized lipophilic cations) which can preferentially be localized in mitochondria. Based on DLC, three photosensitizer DLCs-porphyrin conjugates may include a core modified porphyrin-rhodamine B cation, a core modified porphyrin-mono-triphenyl phosphonium cation, and a core modified porphyrin-di-tPP cation.

Chemical modifications for more accurate targeting have led to the discovery of the next generation photosensitizers, for example 5,10,15,20-Tetrakis(3-hydroxyphenyl)chlorin (mTHPC, Temoporfm). Based on these characteristics, considerable efforts have been devoted to develop specific carriers for delivery of photosensitizers in order to avoid phototoxicity to normal tissues, such as skin.

An example photosensitizer, 5-ALA, is an endogenous compound (approximately 600 mg/day is synthesized by the body) that serves as a precursor in the heme synthetic pathway where 5-ALA is enzymatically converted to protoporphyrin IX (PPIX). PPIX is photoactive and is fluorescent when exposed to long ultraviolet waves. Malignant glial cells are capable of producing PpIX from 5-ALA and the exogenous administration of 5-ALA leads to selective accumulation of PpIX in tumor cells.

It is known that 5-ALA, a prodrug, is metabolized intracellularly to form a fluorescent molecule PPIX. Currently this enables fluorescent guided resection surgeries, wherein administration of 5-ALA leads to preferential accumulation of PPIX in tumor cells and following exposure to blue light (λ=375-410 nm). When exposed to blue light PPIX emits a red-violet fluorescence, distinguishing tumor cells to guide maximal surgical resection. This phenomenon appears to be restricted to high grade tumors; conversely, in low-grade tumors (WHO grade I/II, medulloblastoma, oligodendroglioma) fluorescence is not observed after administration of 5-ALA. Explanations for higher 5-ALA uptake in malignant cells may include a disrupted blood-brain barrier, increased neo-vascularization, and overexpression of membrane transporters.

Without being bound by any theory, it is believed that the mechanism of action for PDT is as follows. 5-Aminolevulinic acid hydrochloride (5-ALA) occurs endogenously as a metabolite that is formed in the mitochondria from succinyl-CoA and glycine. Exogenous administration of 5-ALA leads to accumulation of the 5-ALA metabolite Protoporphyrin IX (PPIX) in tumor cells.

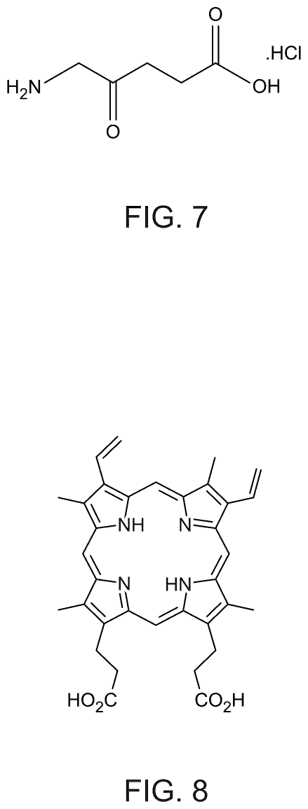

FIG. 7 shows the structural formula for 5-ALA HCL.

FIG. 8 shows the structural formula for PPIX. When illuminated with 375 to 440 nm light, tumor tissue with high concentration of PPIX can be visualized as red fluorescence. Tissue lacking sufficient PPIX concentrations appears blue. When tumor tissue containing high concentrations of PPIX are excited with 635 nm light, the PPIX molecule enters an excited state. Decay back to the ground state can occur through a type I photochemical reaction that creates destructive free radicals or a type II photochemical reaction that generates highly reactive singlet oxygen and other reactive oxygen species (ROS). Generation of singlet oxygen and ROS is the primary mechanism by which the tumor destroying activity of PDT is achieved. Reaction of singlet oxygen or ROS with tumor manifests in necrosis and apoptosis of tumor cells, as well as in the destruction of tumor vasculature. The short half-life of singlet oxygen (on the order of 40 ns) allows reactions only within a distance of about 20 nM. This allows PDT to primarily damage tumor cells while sparing adjacent normal tissue.

### How RePDT Therapy Works

Photodynamic Therapy (PDT) is a two-stage treatment that involves a photosensitive drug and its activation with light. Generally, the initially bioinert photosensitizer (PS) may be delivered orally, intravenously, intraperitoneally, or topically. As stated above, PSs preferentially accumulate in various tumor tissues and have also been shown to cross the blood brain barrier to accumulate in brain cancers. The PS is non-toxic until activation with light whereupon, in combination with molecular oxygen in the cell, cytotoxic reactive oxygen species (ROS) are generated, leading to cellular destruction. This damage can manifest as necrosis and apoptosis of tumor cells, as well as in the destruction of surrounding tumor vasculature. The short distance of migration for ROSs (e.g., ˜0.02-1 μm) and their short lifespan (e.g., ˜0.04-4 μs) allows PDT to primarily damage tumor cells while sparing adjacent normal tissues. With regards to intensity or total delivered energy, intraoperative use of PDT employ dosages may be in the 10-500 J/cm\hat{ }2 range. This range may be considered on the higher side of the dosage based on the “more is better” philosophy.

5-ALA has been approved for PDT applications in oncology and other diseases (e.g., as an adjuvant treatment modality for esophageal and non-small cell lung cancers, Barrett's esophagus and actinic keratoses amongst others). However, due to limited light access inside the body, the use of PDT has been restricted to treatment of superficial cancers and tissue accessible by endoscopy.

The wirelessly powered illumination implantable devices and systems disclosed herein are designed to overcome at least some of these limitations using an implantable light source that can be wirelessly powered. An implanted light source can be used to activate drugs intraoperatively, and/or post-surgery on demand and noninvasively within the patient. This enables multiple rounds of therapy and long-term treatment, thus establishing a drug regimen. Repeated application of PDT ensures that the rate of tumor cell death in the treatment region exceeds its rate of regrowth, effectively minimizing the chances of cancer cell infiltration and recurrence.

Examples of PDT devices disclosed herein, comprising safe and repeated applications of low-dose PDT in internal tissues, are designed to be a new treatment modality for malignant gliomas. The examples disclosed herein may be implanted in the tumor cavity after tumor resection surgery. The examples disclosed herein do not necessarily require a tumor resection surgery for implantation surgery and may be implanted directly in target tissue. The examples disclosed herein may be used to treat one or more tumors with PDT intraoperatively, post-surgically, or a combination of both to establish a repeated PDT treatment regimen. Follow-up treatment as a post-operative adjunct therapy may be conducted in an outpatient setting or in the clinic. The implant is powered by a custom transmitter device to controllably illuminate the resection cavity at specific wavelengths. The device may be configured to emit red or near-IR light (e.g., 630 nm) inside the cavity of a resected tumor or in any target treatment tissue or organ, at a wavelength that activates the photosensitizer drug. The positioning of the light sources may be optimized to create an illumination zone within the target tissue that will result in photoactivation of a photosensitizing drug to enact PDT. As a result, PDT can be regularly triggered on-demand to suppress any regrowth of remaining or recurrent malignancies in and beyond the margins of the surgical cavity or target tissue. One or more applications of photodynamic therapy induces a robust immune response capable of preventing local tumor recurrence and delaying, and in some cases preventing, the growth of distant, untreated disease (e.g. the abscopal effect).

Examples of treatment methods disclosed herein are believed to be improvements over previous applications of PDT. At least one of these reasons is that previous applications of PDT were largely single use (intra-operative) techniques which may have been an ineffective approach to controlling tumor growth over the long term. Using the rePDT implant and treatment method enables application of multiple rounds of PDT to solid tumors. Repetition permits effective disease control over time.

Additionally, the devices and methods described herein allow application of PDT after surgery, so there is greater freedom for the clinician to use this technique when it is more effective against the cancer and safer for the patient. For example, post-recovery, when the patient is not on immunosuppressives, there is greater potential synergy of PDT with the immune system to elicit secondary protective effects.

Moreover, the implantable light has been designed to be “photosensitizer agnostic,” meaning the device can be paired with next generation photosensitizers as they are developed. With adaptations for anatomical placement and use of the optimum photosensitizer for a particular tumor, this approach is a platform for the treatment of many solid cancers. For example, a patient may receive repeated PDT therapy by being administered a dose of photosensitizer drug (in some cases, 20 mg/kg) that will be activated by light. In one example of a regimen, patients will be administered the photosensitizer and receive PDT on days 14, 28, 42, and 56. The Patient will be monitored by MRI and will receive additional PDT at regularly scheduled intervals. Patients may also receive PDT upon detection of recurrence.

### GBM Models of Disease Demonstrate Efficacy of RePDT

Several models were used to demonstrate the efficacy of repeated photodynamic therapy. The following sections will discus the use of an in vitro testing model, and mouse GBM model.

### In Vitro Testing

An in vitro model of light penetration into tissue was established to determine the quantitative relationship between light penetrance PDT instigated cell death. The primary parameters for light input are illumination intensity and duration of illumination. The primary parameter for PDT instigated cell death is depth (or distance from the light source to the target treatment tissue), which directly attenuates light penetration into tissue.

To evaluate PDT for treatment of gliomas and brain cancers glioma cells were propagated in a hydrogel medium in a three-dimensional (3D) culture to mimic the cellular and optical environment of the brain. A commercial 3D culture system was used to set up the culture in a linear configuration such that a point source of light could be applied to one end of a closed culture in a cylinder form. The total cellular growth volume was 1 mm×1 mm×15 mm. A 630 nm LED light source that mimics the spectral characteristics and intensity of the any of the implantable devices disclosed herein was used to illuminate embedded glioma cells and to establish the effective PDT “kill radius.”

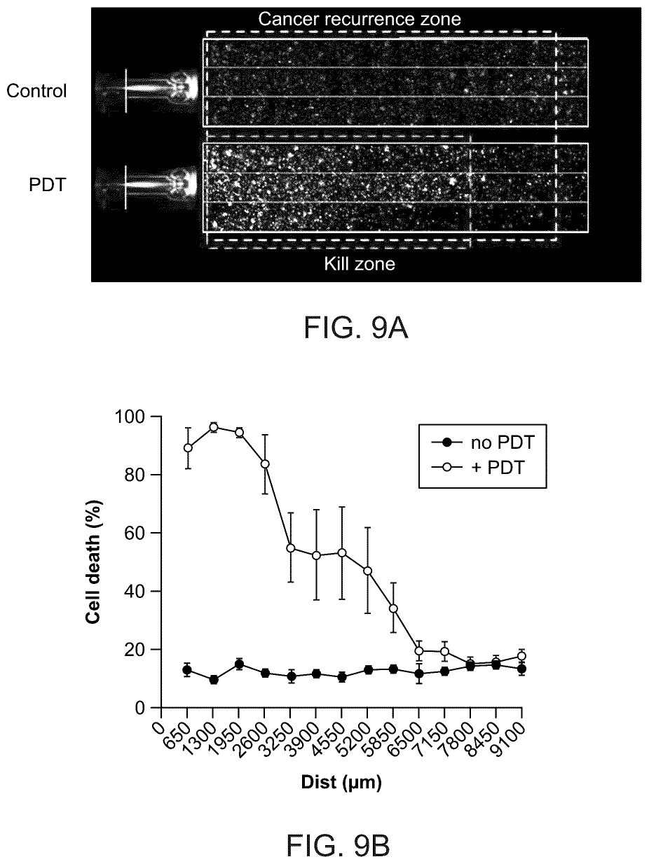

FIGS. 9A-9B show PDT induced cell death of glioma cells using a LED light source such as any of those disclosed herein. Glioma cells were embedded in a 3D-hydrogel, incubated with study drug 5-ALA, and illuminated with a light from a LED at 630 nm as shown in FIG. 9A. The 3D culture system was stained for nuclei (violet) or cell death (red) after PDT treatment. The effective “kill zone” was 6-8 mm from the light source, although lower amounts of cell death were observed at greater distances as seen in FIG. 9B. Cell death (%) vs distance from light source is therefore demonstrated in FIGS. 9A-9B for a representative replicate.

These results indicate that illumination of brain tissue by a point source of 630 nm light can result in PDT mediated cellular death of glioma tissue, with a maximum measured effective range of 6-8 mm. Given that 80% of glioblastoma recurrences occur within the 1 cm resection margins of a primary tumor, this study highlights the potential for PDT to serve as an effective means of supressing tumor growth within this high risk area. This data also confirms linear dose response vs time and light intensity exists. It also supports the repeated PDT thesis that lower intensity, longer time, and repeated doses do in fact improve results, patient outcomes, and progression free survival.

### Rat Xenograft GBM Models

To enable pre-clinical testing of illumination device prototypes such as those disclosed herein, the use of a Sprague Dawley rat model of GBM was established.

C6 rat glioma cells were maintained in F12K medium (F-12K Nutrient Mixture, 1X, Gibco) supplemented with 10% 2.5% FBS (Biowest), 2.5% 15% Horse Serum (Gibco), and 1% each of P/S and L/G antibiotics (Biowest) at 37° C. in a humidified incubator containing 5% CO₂. Subconfluent, low passage C6 glioma cells were resuspended to single cell suspension in sterile PBS (1X, Hyclone) within two hours of xenotransplantation.

Approximately 2×10⁵ cells were stereotactically injected into the striatum of rats (230-270 g, InVivos Pte Ltd) under general anaesthesia. Using a hand-held drill, a burr hole of 1 mm was drilled and cells were injected unilaterally at 3 mm lateral to Bregma and at a depth of 4 mm. Injections were conducted using borosilicate capillary glass micropipettes (A-M Systems) pulled to a tip diameter of ˜10 mm. Animals were allowed to recover with daily antibiotics and analgesics for up to a week and monitored post operatively for adverse events and signs of distress in compliance with relevant animal treatment guidelines.

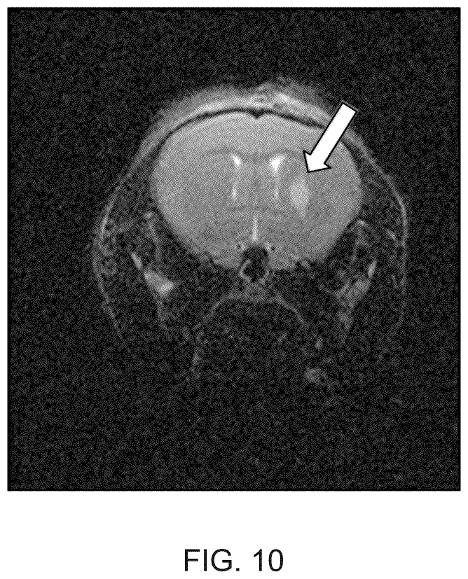

Tumor engrafting and growth typically followed a rapid progression, allowing interventional studies on disease progression. Engrafted tumors exhibited morphology consistent with the C6 glioma model and is representative of human high-grade gliomas as seen by the arrow in FIG. 10.

FIG. 10 illustrates an orthotopic xenograft model. Rat C6 glioma cells are implanted unilaterally into the striatum of Sprague Dawley rats. Tumor engraftment and growth was rapid and morphology is consistent with human high grade gliomas.

H&E (hematoxylin & eosin) staining shows aggressive neoplastic growth in the area surrounding the injection site. The pattern exhibits proliferation at the periphery of the tumor with a necrotic core, consistent with the morphology of an encapsulated high-grade glioma. The tumor stains positive for the cell proliferation marker Ki67 and takes up the photosensitizer drug 5-ALA. Strong PPIX signal in the tumor indicates conversion of 5-ALA to PPIX, the photosensitive and cytotoxic substrate for PDT.

### Survival Analysis for Xenograft and PDT Experiments

Animals were monitored daily for weight change and behavioral abnormalities ahead of xenograft induced mortality. Ethical termination via CO₂ overdose was triggered in the following events: individual weight loss of greater than 20% in one day, extended cessation of motor behavior and loss of grooming; paralysis and lack of movement/lying on a side. After termination or death, individual rat brains were dissected and prepared for histological analysis by freezing in OCT compound (Tissue-Tek). Survival analysis was performed via the method of Kaplan-Meier.

### Single Application of PDT Treatment

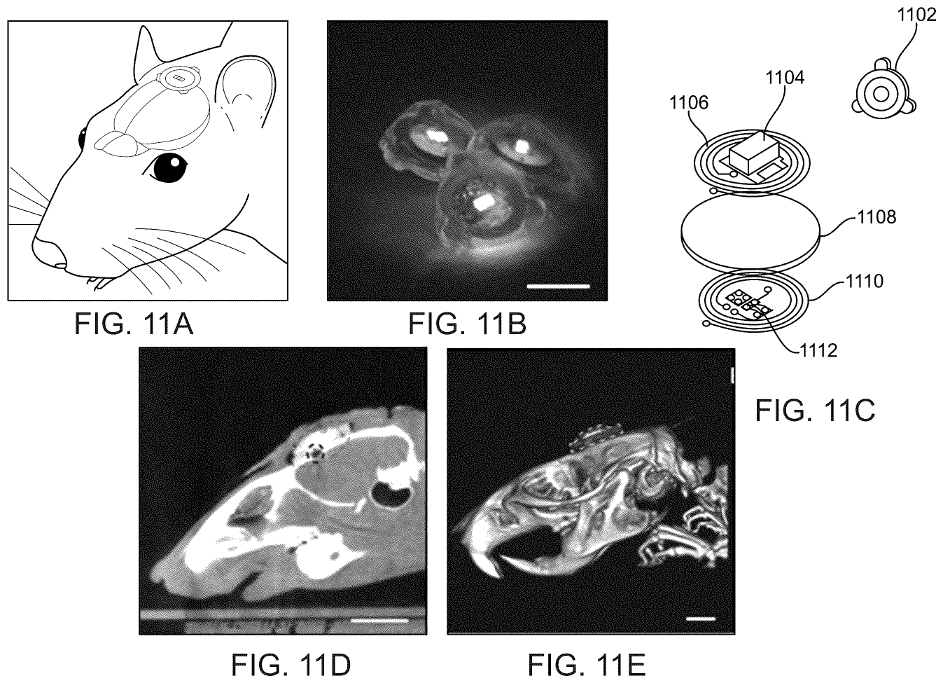

To enable PDT treatment, light devices such as any of those disclosed herein, were implanted at the time of xenograft surgery and were placed atop the cranium with the LED element situated above the injection burr hole as seen in FIG. 11A. The implants were secured in place by suturing the overlying skin to close the surgical site. Device operation was tested briefly before closure. Six hours prior to each PDT treatment, rats were injected with 100 mg/kg 5-ALA (Synchem UG & Co. KG) prepared in sterile PBS (1X, Hyclone) via tail vein or with a vehicle control. PDT treatment was conducted under 1.5-2.5% halothane isoflurane/O₂ general anesthesia. Wireless powering coils such as those disclosed herein were secured to each animal's head, and each PDT treatment delivered 2.5 mW/cm2 of light over 3,600 seconds for a total fluence of 9 J/cm² per treatment. Any of the other treatment parameters disclosed herein may be used.

Test animals were sacrificed after 24 hours, and their brains were sectioned for histological examination. The proliferation marker Ki67 and the TUNEL assay (terminal deoxynucleotidyl transferase dUTP nick end labeling) indicative of apoptosis was used to quantify the PDT effect. Wirelessly powered light delivery in combination with 5-ALA was able to induce PDT, whereas treatment with either component singly did not show the same effect.

FIGS. 11A-11E show implantable illumination devices such as any of those disclosed herein used to treat GBM in rodents.

FIG. 11A shows a schematic of PDT treatment in an orthotopic model of GBM. Tumor grafts are surgically implanted in the rat striatum. The LED device is implanted surgically atop the skull and positioned to directly illuminate the tumor graft site. 5-ALA PDT treatment is applied to elicit local destruction of the tumor.

FIG. 11B is an image of wireless LED devices emitting light.

FIG. 11C is a schematic of an implantable illumination device and electrical components. The device in FIG. 11C may be any of the devices disclosed herein, and may include light source 1104 such as an LED, coils 1106, 1110, printed circuit board (PCB) 1108, a silicone cap 1102 and electronic components 1112 such as a capacitor, diode or any other electronic component needed to operate the device.

FIGS. 11D-11E show a CT scan of an LED device positioned in a test animal showing approximate positioning of the LED light element relative to the tumor graft site.

Test animals were sacrificed after 24 hours, and their brains were sectioned for histological examination. The proliferation marker Ki67 and the TUNEL assay indicative of apoptosis was used to quantify the PDT effect. Wirelessly powered light delivery in combination with 5-ALA was able to induce PDT, whereas treatment with either component singly did not show the same effect.

### Single vs. Repeated PDT (rePDT) Treatment

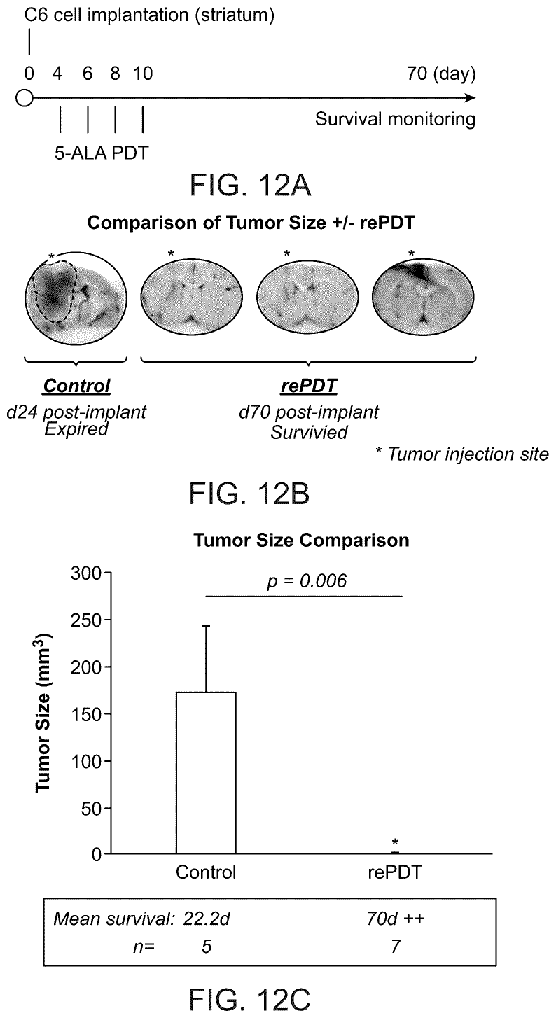

Further experimentation was conducted to determine if repeated application of PDT treatment would have a cumulative effect on tumor growth and disease progression. In vivo, C6 cells exhibit a doubling time of approximately 2.6 days. If a single PDT treatment was at least 50% effective in destroying the tumor population, an inter-treatment interval of 2 days would maintain or diminish the tumor graft to at, or near starting levels. With this rationale in mind, PDT treatment was applied according to the schema shown in FIG. 12A with four treatments repeated every two days starting at day 4 after xenograft surgery.

FIGS. 12A-12C show a PDT treatment schema and survival in an orthotopic GBM model.

FIG. 12A shows a treatment schema where C6 glioma cells were injected into rat striatum on day 0. On that same day, devices were surgically implanted above the injection site.

FIG. 12B shows representative images of harvested rat brains that received control treatment (single PDT session) and those that received repeated PDT treatments. Dotted line indicates tumor location.

FIG. 12C shows mean survival times of the control group (n=5) and the repeated PDT group of (n=7) animals.

Similar to single treatment PDT experiments, C6 tumor xenografts were stereotactically and unilaterally injected into the striatum of Sprague Dawley test rats (FIG. 12B) and wireless devices such as those disclosed herein were positioned subcutaneously atop the skull, directly superior to the tumor injection site. On the day of treatment, animals received 5-ALA via tail vein injection and allowed to recover for 6 hours to allow uptake and expression of PPIX. PDT treatment was conducted under light general anaesthesia via halothane to reduce movement during PDT. All test animals recovered with no adverse effects. This procedure was repeated at 2-day intervals for a total of four treatments.

Control animals exhibited robust tumor treatment progression and exhibited rapid mortality between 22-24 days, consistent with previous reports of C6 glioma in orthotopic models. Upon necropsy, control animals exhibited large infiltrative tumors which displaced native brain tissue leading to mortality. In contrast, animals receiving repeated PDT (rePDT) survived with no adverse symptoms or behavior until the termination of the experiment at day 70 (FIG. 12C). When examined morphologically and histologically, the brains exhibited no significant tumor tissue graft progression. Gross estimates of tumor size between controls and rePDT treated animals are shown in FIG. 12C. Control animals displayed large tumors, ranging in size from 125-200 mm³. Tumor diameters of <5 mm often impinged on brain structures critical for homeostasis such as ventral medulla and was a likely cause of death. In contrast, rePDT brain sections were largely devoid of remaining tumor cells. The estimates of the tumor size are based upon scar tissue and necrotic artifacts from the xenograft injection scar in the brain. By these estimates, there is a >99.5% reduction in the tumor volume due to rePDT treatment. Trace levels of remaining tumor, however small, would be able to grow into a significant tumor volume. Therefore, the animals were monitored for an additional 50 days after the median survival time of the control animals. Fifty days is 2× the median survival time, which would allow for double the period of exponential growth needed to induce mortality in a normal xenograft of 200,000 cells.

Histological analysis of rePDT animals show trace amounts of tumor cells by H&E staining and low levels of Ki67 (not significantly above background when compared with contralateral controls).

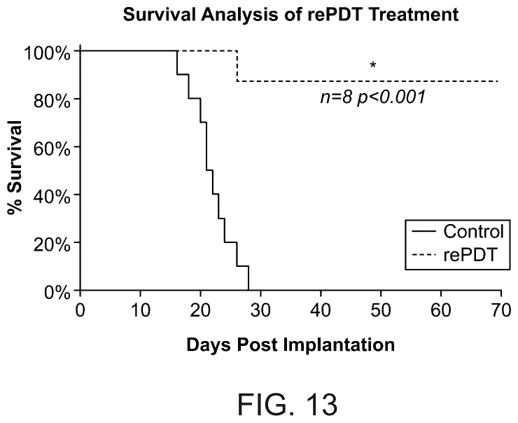

FIG. 13 shows the survival analysis of the control vs rePDT populations conducted via the Kaplan-Meier method. The median survival of control animals (n=10) is 21 d, whereas 8 out of 9 rePDT animals (89%) were living at the end of the experiment. This correlates with p<0.001 suggesting that the rePDT treatment resulted in a significant interventional step for animal survival.

FIG. 13 also illustrates survival curves for single vs repeat PDT treated animals. Sprague Dawley rats were implanted with c6 glioma cells and implanted with a wirelessly powered light source above the injection site as described. On treatment days (days 4, 6, 8, 10), animals were given the study drug 5-ALA as described and received PDT treatment. Cohort 1 animals, which received treatment only on day 4, had a mean survival ˜22 days (n=10). In contrast, 7 of 8 cohort 2 animals, which received PDT treatment on days 4, 6, 8, and 10, survived to day 70 at which point the experiment was stopped. Therefore, rePDT kills more of the tumor compared with a single treatment of PDT and increases survival time of the rodents.

### MRI Compatability

Comparison of tumor size progression, cell density and morphology reveals that rePDT can continuously suppress tumor progression and induce tumor cell death without negatively affecting the surrounding normal tissue.

To determine compatibility of implantable illumination devices such as those described herein with MRI, a test-device was implanted into an adult human cadaver head and imaged in a 1.5 T MR scanner.

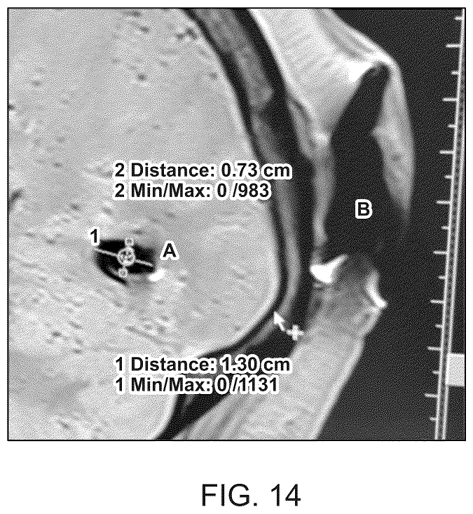

FIG. 14 shows a partial sagittal slice of human cadaver brain and demonstrates that MR imaging shows little to no distortion from the light source portion of the device. In this image, an example of an illumination device has been implanted after mock tumor debulking surgery. The 7×13 mm dimension of the light source (marked “A” in FIG. 14) is clearly visible with minimal distortion or voids around it. The titanium case containing electronics (marked “B” in FIG. 14) is visible posterior to the skull and just under the skin, and does show minimal distortion, which is a mere artifact given the limitations of the cadaver specimen. A neuro-radiologist reviewed the data and advised that this device would likely not interfere with MRI monitoring of GBM patients after a resection surgery. Therefore, the implant will not interfere with MRI scans. The current GBM approach is a combination of surgical resection, radiotherapy and Temozolomide (TMZ) chemotherapy, however, there are cases in which GBM were unresectable or developed resistance towards the standard treatment. Hence, there is an urgent need for developing novel approaches for GBM treatment. PDT is an increasingly popular two-stage treatment scheme that combines light energy with photosensitizers. By exploiting the characteristics of photosensitizers to accumulate in tumor cells, PDT can selectively induce tumor cell death by generating reactive oxygen species.

PDT has been shown to prolong survival in GBM patients during clinical trial. However, these applications were confined to sPDT, which only expressed limited anti-tumor effect. Hence, the present devices and methods encourage the use of rePDT, by showing that rePDT can exert continuous anti-tumor effect on GBM, and monitor treatment outcomes using MRI.

In this study, 5-ALA was chosen due to the rapid systemic clearance of 5-ALA-induced PPIX within 24 hours. This allows rePDT to be carried out every 48 hours, which is how intervals of rePDT were established. Other time intervals may be used such as every 12, 24, 36, 48, 60, 72 hours, or longer if desired. 5-ALA dosage was set to be 250 mg/kg, which may be translated into a human equivalent dose of around 20 mg/kg, as this dose was widely used in clinical trials, it is easier to translate the current findings into clinical applications. Other dosages may also be used such as 5, 10, 15, 20, 25, 30, 35, 40, 45, or 50 mg/kg as desired. The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

### NOTES AND EXAMPLES

The following, non-limiting examples, detail certain aspects of the present subject matter to solve the challenges and provide the benefits discussed herein, among others.

Example 1 is a method for phototherapy, comprising: delivering a photosensitizer to a patient, wherein the photosensitizer accumulates in a target treatment tissue; illuminating the target treatment tissue with light from an illumination source in a wirelessly powered illumination device; photoactivating the photosensitizing agent with the light wherein in combination with oxygen in cells of the target treatment tissue, cytotoxic reactive oxygen species (ROS) are generated; and causing cells in the target treatment tissue to be destroyed by the cytotoxic reactive oxygen species.

Example 2 is the method of Example 1, wherein delivering the photosensitizer comprises delivering the photosensitizer orally, intravenously, intraperitoneally, or topically to the patient.

Example 3 is the method of any of Examples 1-2, wherein the photosensitizing agent comprises one or more of benzoporphyrins, purpurins, texaphyrins, phthalocyanines, naphthalocyanines, and protoporphyrin IX (PPIX).

Example 4 is the method of any of Examples 1-3, wherein the photosensitizing agent comprises 5-aminolevulinic acid (5-ALA).

Example 5 is the method of any of Examples 1-4, wherein the target treatment tissue comprises a tumor.

Example 6 is the method of any of Examples 1-5, wherein the target treatment tissue comprises a glioblastoma multiforme tumor.

Example 7 is the method of any of Examples 1-6, further comprising: discontinuing the illuminating for a first desired period of time; administering a new dose of the photosensitizer to the patient; and re-starting the illuminating for a second desired period of time.

Example 8 is the method of any of Examples 1-7, wherein repeating the discontinuing of illumination and the re-starting of the illumination repeatedly results in destruction of more cells in the target treatment tissue than a single treatment of photodynamic therapy.

Example 9 is the method of any of Examples 1-8, wherein the illumination source is no more than 1 cm away from the target treatment tissue.

Example 10 is the method of any of Examples 1-9, further comprising implanting the illumination source in a brain of the patient.

Example 11 is the method of any of Examples 1-10, wherein the illumination source is no more than 6-8 mm away from the target treatment tissue.

Example 12 is the method of any of Examples 1-11, wherein the light has a wavelength in the near infrared region of the spectrum.

Example 13 is the method of any of Examples 1-12, wherein cell destruction in the target treatment tissue follows a linear dose response to time and light intensity of the illumination from the illumination source.

Example 14 is the method of any of Examples 1-13, further comprising destroying tumor vasculature with the reactive oxygen species.

Example 15 is the method of any of Examples 1-14, wherein the reactive oxygen species causes apoptosis and necrosis of the target treatment tissue.

Example 16 is the method of any of Examples 1-15, further comprising monitoring the target treatment tissue with magnetic resonance imaging (MRI).

Example 17 is the method of any of Examples 1-16, further comprising personalizing a treatment regimen for the patient by modifying the delivering of the photosensitizer, or modifying the illuminating of the target treatment tissue, or modifying the photoactivating of the photosensitizer agent, based on the monitoring of the target treatment tissue with magnetic resonance imaging.

Example 18 is the method of any of Examples 1-17, further comprising inducing an anti-tumor immune response in the patient, and preventing local tumor growth in the target treatment tissue or delaying growth of distant untreated disease away from the target treatment tissue, after one or more applications of photodynamic therapy to the patient.

Example 19 is a device for illuminating target treatment tissue, comprising: an illumination source configured to be disposed adjacent the target treatment tissue; a power receiver element configured to receive power wirelessly from an external power source; a housing containing electronic components configured to control operation of the illumination source; and a tether coupling the illumination source with the electronic components in the housing.

Example 20 is the device of Example 19, wherein the illumination source comprises a LED configured to emit near infrared light.

Example 21 is a system for illuminating target treatment tissue, comprising: the device of any of Examples 19-20, and an external power source configured to wirelessly provide power to the illumination source.

Example 22 is the system of any of Examples 19-21, further comprising a burr hole cap or securement plate configured to close a burr hole in a skull of a patient and configured to help secure at least a portion of the device to the skull.

Example 23 is the system of any of Examples 19-22, further comprising a headband configured to hold the external power source adjacent the power receiver element so that power can be wirelessly transmitted from the external power source to the power receiver element.

Example 24 is the system of any of Examples 19-23, further comprising a photosensitizing agent, wherein light from the illumination source is configured to photoactivate the photosensitizing agent.

Example 25 is the system of any of Examples 19-24, wherein the photosensitizing agent comprises 5-aminolevulinic acid (5-ALA).

In Example 26, the devices, systems, or methods of any one or any combination of Examples 1-25 can optionally be configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A method for phototherapy, comprising:
delivering a photosensitizer to a patient, wherein the photosensitizer accumulates in a target treatment tissue;
illuminating the target treatment tissue with light from an illumination source in a wirelessly powered illumination device;
photoactivating the photosensitizing agent with the light wherein in combination with oxygen in cells of the target treatment tissue, cytotoxic reactive oxygen species (ROS) are generated; and
causing cells in the target treatment tissue to be destroyed by the cytotoxic reactive oxygen species.

2. The method of claim 1, wherein delivering the photosensitizer comprises delivering the photosensitizer orally, intravenously, intraperitoneally, or topically to the patient.

3. The method of claim 1, wherein the photosensitizing agent comprises one or more of benzoporphyrins, purpurins, texaphyrins, phthalocyanines, naphthalocyanines, and protoporphyrin IX (PPIX).

4. The method of claim 1, wherein the photosensitizing agent comprises 5-aminolevulinic acid (5-ALA).

5. The method of claim 1, wherein the target treatment tissue comprises a tumor.

6. The method of claim 1, wherein the target treatment tissue comprises a glioblastoma multiforme tumor.

7. The method of claim 1, further comprising:
discontinuing the illuminating for a first desired period of time;
administering a new dose of the photosensitizer to the patient; and
re-starting the illuminating for a second desired period of time.

8. The method of claim 7, wherein repeating the discontinuing of illumination and the re-starting of the illumination repeatedly results in destruction of more cells in the target treatment tissue than a single treatment of photodynamic therapy.

9. The method of claim 1, wherein the illumination source is no more than 1 cm away from the target treatment tissue.

10. The method of claim 1, further comprising implanting the illumination source in a brain of the patient.

11. The method of claim 1, wherein the illumination source is no more than 6-8 mm away from the target treatment tissue.

12. The method of claim 1, wherein the light has a wavelength in the near infrared region of the spectrum.

13. The method of claim 1, wherein cell destruction in the target treatment tissue follows a linear dose response to time and light intensity of the illumination from the illumination source.

14. The method of claim 1, further comprising destroying tumor vasculature with the reactive oxygen species.

15. The method of claim 1, wherein the reactive oxygen species causes apoptosis and necrosis of the target treatment tissue.

16. The method of claim 1, further comprising monitoring the target treatment tissue with magnetic resonance imaging (MRI).

17. The method of claim 16, further comprising personalizing a treatment regimen for the patient by modifying the delivering of the photosensitizer, or modifying the illuminating of the target treatment tissue, or modifying the photoactivating of the photosensitizer agent, based on the monitoring of the target treatment tissue with magnetic resonance imaging.

18. The method of claim 1, further comprising inducing an anti-tumor immune response in the patient, and preventing local tumor growth in the target treatment tissue or delaying growth of distant untreated disease away from the target treatment tissue, after one or more applications of photodynamic therapy to the patient.

19. A device for illuminating target treatment tissue, comprising:
an illumination source configured to be disposed adjacent the target treatment tissue;
a power receiver element configured to receive power wirelessly from an external power source;
a housing containing electronic components configured to control operation of the illumination source; and
a tether coupling the illumination source with the electronic components in the housing.

20. The device of claim 19, wherein the illumination source comprises a LED configured to emit near infrared light.

21. A system for illuminating target treatment tissue, comprising:
the device of claim 19, and
an external power source configured to wirelessly provide power to the illumination source.

22. The system of claim 21, further comprising a burr hole cap or securement plate configured to close a burr hole in a skull of a patient and configured to help secure at least a portion of the device to the skull.

23. The system of claim 21, further comprising a headband configured to hold the external power source adjacent the power receiver element so that power can be wirelessly transmitted from the external power source to the power receiver element.

24. The system of claim 21, further comprising a photosensitizing agent, wherein light from the illumination source is configured to photoactivate the photosensitizing agent.

25. The system of claim 24, wherein the photosensitizing agent comprises 5-aminolevulinic acid (5-ALA).
